(19) United States
(12) Patent Application Publication
(10) Pub. No.: US 2003/0190355 A1
(43) Pub. Date: Oct. 9, 2003

(54) Modified release minerals

(76) Inventors: Marc S. Hermelin, St. Louis, MO (US); Michael Grimshaw, St. Louis, MO (US)

Correspondence Address: NATH & ASSOCIATES PLLC, Sixth Floor, 1030 15th Street, N.W., Washington, DC, 20005, US

(21) Appl. No.: 10/115,892

(22) Filed: Apr. 5, 2002

(51) Int. Cl. A61K 9/22

(52) U.S. Cl. 424/468

(57) ABSTRACT

The present invention is directed to compositions and methods for the enhancement of iron uptake or the treatment of iron deficiency by enhancing the rate and extent of dissolution in a subject in need thereof. The composition contains at least two iron-providing materials in a single dosage form wherein at least one of the iron-providing materials contains a modified release mechanism, matrix, or coating. The iron-providing materials included within the composition have different rates of release. Following administration to the animal, the iron-providing materials are released in the gastrointestinal tract over a period of up to 24 hours.

### BACKGROUND OF THE INVENTION

1. Field of the Invention

The present inventive subject matter is directed to the enhancement of iron uptake or the treatment of iron deficiency, and in particular to modified release iron compositions which contain combinations of at least two iron-providing substances for maximizing the absorption of iron.

2. Description of the Related Art

Ionic iron is an essential metal for the growth and maintenance of animals and most microorganisms. In humans, iron is required for oxygen metabolism, hemoglobin production, and electron transfer reactions, among many other reactions. Ionic iron is actively transported to the mucosal cells of the intestine, where it binds to the protein ferritin. This phenomenon is called the mucosal iron barrier. The iron-ferritin complex then serves as a local intracellular storehouse for iron. When body reserves of iron are adequate, very little iron is allowed to pass into the portal blood, and most of the stored iron is lost as the epithelial cells later slough off. As iron reserves are depleted, as occurs, for example, during acute or chronic hemorrhage, iron uptake from the intestine and its release to the blood are accelerated.

An adequate supply of iron to the body is an essential requirement for tissue growth in both humans and other animals. Although there is normally an ample amount of iron in the diet, the level of absorption of iron from food is generally low so that the supply of iron to the body can easily become critical under a variety of conditions.

Iron deficiency anemia is commonly encountered in pregnant and lactating women. Moreover, in certain pathological conditions there is a malabsorption or maldistribution of body iron leading to a state of chronic anemia. Such malabsorption or maldistribution is seen in certain intestinal disorders, chronic diseases such as rheumatoid arthritis, certain haemolytic diseases, and cancer. Iron deficiency also results in gastrointestinal problems, anorexia, and paresthesia. Anemia is usually treated with a combination therapy of diet, iron supplements, and additional vitamins, such as vitamin B-12 and folic acid, to increase the absorption of iron. Pregnant women in particular are most often provided with iron supplements to guard against iron-deficiency.

The absorption of iron supplements from the gastrointestinal tract is reduced in the presence of divalent non-iron mineral supplements such as magnesium and calcium. See Goodman et al., the Pharmacological Basis of Therapeutics, Third Edition, page 1396 (1968); Freeman et al., Am. J. Physiol., 137, p. 706-9 (1942); and Amine et al., N. Nutrition, 101, p. 927-936 (1971), each of which is hereby incorporated by reference in their entirety. Many prenatal iron supplement compositions described in the literature accordingly contain limited quantities of calcium and/or magnesium, e.g. Filibon® prenatal capsules, PDR 20th Ed., p. 670 (1965) and U.S. Pat. No. 4,431,634, each of which is hereby incorporated by reference in their entirety.

Although a wide range of iron compounds is marketed for the treatment of iron deficiency, the results thereof, and the prophylaxis of such iron-deficiency states, the level of iron uptake by the body from these compounds is often quite low, necessitating the administration of relatively high dosage levels of the compound. The administration of a high dose of poorly absorbed iron complexes may cause siderosis of the gut wall and a variety of side effects such as nausea, vomiting, constipation, diarrhea, abdominal discomfort, shortness of breath, weight loss, headaches, dizziness, anorexia, fatigue, darkened urine, heartburn, darkened teeth, darkened stool, and heavy malodorous stools.

It is therefore one aspect of the present invention to provide a means for enhancing the rate and extent of absorption of iron. It has been found that this may be achieved through the administration of a combination of different iron substances, including a modified release dosage form. Through this mechanism, at least two iron substances are provided, each substance having a different rate of release. Accordingly, the instant combination provides both an initial absorption of iron and a prolonged absorption of iron over time upon administration to a patient.

A modified release dosage form remedies some of the aforementioned problems because it can deliver the same amount of biologically active substance in one dose that an immediate release dosage form delivers in many doses. A modified release composition is one that achieves slow release of a biologically active substance over an extended period of time and extends the duration of action over the action achieved by conventional delivery. A modified release drug delivery system provides several advantages over an immediate release drug delivery system, such as reduction of the frequency of administration and the maintenance of effective concentrations of the biologically active substance in the blood. The maintenance of an effective concentration in the blood reduces the chance of side effects, such as nausea, and toxicity as occurs if too much of the biologically active substance is concentrated in the blood at any one time. The use of a modified release drug delivery system lessens the amount of iron administered to a patient, thereby reducing the chances the patient will experience the side effects mentioned above. This benefit is especially important when a toxic amount of a biologically active substance can cause illness and death.

Additionally, by using a modified release drug delivery system, it is not necessary to overload the carrier with iron for administration. This prevents the undesired waste of overloaded iron included in immediate release dosage forms which can not be processed by the body.

Another advantage to using at least two different iron-providing materials, each having a different release rate, is discovered when one considers that these materials are delivered to the stomach. As the gastric environment changes between fasted to fed states, the dissolution of a drug, vitamin, or mineral, and subsequent absorption in the small intestine, will vary, sometimes significantly. Each of these states changes the solubilization of the iron. Accordingly, it is advantageous to administer different iron sources having different solubility profiles.

Numerous approaches for administering extended and controlled release formulations have been described in various references. For example, Busetti et al., U.S. Pat. No. 5,891,474, hereby incorporated by reference in its entirety, discloses a method of achieving a time specific delivery of a pharmaceutically active agent to a patient. The pharmaceutical agent is encased in a swellable polymeric coating which delays the release of the pharmaceutical agent for a predetermined period of time depending on the thickness of the polymeric coating.

Baichwal et al., U.S. Pat. No. 5,662,933, hereby incorporated by reference in its entirety, discloses an extended release pharmaceutical formulation. The extended release formulation includes a gelling agent, an inert pharmaceutical diluent, a hydrophobic material and/or coating, and a medicament for extended oral administration.

Goldie et al., U.S. Pat. No. 4,990,341, hereby incorporated by reference in its entirety, discloses a solid, controlled release oral dosage form. The dosage form comprises a therapeutic amount of hydromorphone or salt in a matrix for use with moderate to severe pain. The dosage is formulated such that the peak plasma level of hydromorphone is attained at 2-4 hours following administration of the dosage form.

Acharya, U.S. Pat. No. 5,686,094, hereby incorporated by reference in its entirety, discloses a controlled release formulation for the treatment of xerostomia. The delivery system comprises a polycarbophil coating surrounding an active agent. The coating system may also be used with cosmetics and household items where a controlled release effect is beneficial.

Yang et al., U.S. Pat. No. 5,576,022, hereby incorporated by reference in its entirety, discloses a controlled release tacrine drug delivery system comprising a sustained release composition and a controlled release composition wherein the controlled release composition is present at specific ratios to the immediate release composition.

It is a continuing goal of researchers to develop pharmaceuticals which can regulate the levels of cellular iron and thus treat iron deficient conditions and enhance iron uptake in a patient.

### SUMMARY OF THE INVENTION

The present inventive subject matter relates to a composition for the enhancement of iron uptake or the treatment of iron deficiency by enhancing the rate and extent of iron dissolution in a subject in need thereof, which comprises: a combination of at least two iron-providing materials in a single dosage form;

wherein at least one of the iron-providing materials has a modified release mechanism, matrix, or coating;

wherein each of the iron-providing material has a different rate of release; and

wherein the iron-providing materials are released in the gastrointestinal tract over a period of up to 24 hours.

The present inventive subject matter additionally relates to a composition for the enhancement of iron uptake or the treatment of iron deficiency in a mammal in need thereof, which comprises: a combination of at least two iron-providing materials in a single dosage form;

wherein at least one of the iron-providing materials has a modified release mechanism, matrix, or coating;

wherein each of the iron-providing material has a different rate of release; and

wherein the iron-providing materials are released in the gastrointestinal tract over a period of up to 24 hours.

Another embodiment of the present inventive subject matter is a method for the enhancement of iron uptake or the treatment of iron deficiency in a subject in need thereof, which comprises: administering to said subject a therapeutically effective amount of at least two iron-providing materials combined in a single dosage form;

wherein at least one of the iron-providing materials has a modified release mechanism, matrix, or coating;

wherein each of the iron-providing material has a different rate of release; and

wherein the iron-providing materials are released in the gastrointestinal tract over a period of up to 24 hours.

Yet another embodiment of the present inventive subject matter is a method for the enhancement of iron uptake or the treatment of iron deficiency in a subject in need thereof, which comprises: administering to said subject a therapeutically effective amount of at least two iron-providing materials having different solubilities combined in a single dosage form;

wherein the iron-providing materials naturally have different rates of release; and

wherein the iron-providing materials are released in the gastrointestinal tract over a period of up to 24 hours.

### DETAILED DESCRIPTION OF THE INVENTION

As used herein, “animal” refers to a human, mammal, or any other animal.

As used herein, “mammal” refers to a warm-blooded vertebrate animal, including humans, characterized by a covering of hair on the skin.

“Biologically active substance” refers to any substance or substances comprising a drug, active therapeutic substance, metabolite, medicament, vitamin, or mineral; any substance used for treatment, prevention, diagnosis, cure, or mitigation of disease or illness; any substance which affects anatomical structure or physiological function; or any substance which alters the impact of external influences on an animal, or metabolite thereof, and as used herein encompasses the terms “active substance”, “therapeutic substance”, “agent”, “active agent”, “drug”, “medication”, “medicine”, “medicament”, and other such similar terms.

As used herein, a “modified release” when applied to the formulation's mechanism, matrix, or coating means a release rate that is different from the materials normal release rate, namely a system that may be modified to achieve a delayed, sustained, controlled, or extended release in comparison to a compound's normal release rate.

The present inventive subject matter provides a composition for the enhancement of iron uptake or the treatment of iron deficiency in an animal, as well as methods of using the same. The animal may be a human. Furthermore, the human may be an adult. Alternatively, the human may be a child. The composition contains a plurality of iron-providing materials, minimally at least two, in a single dosage form. More than two iron-providing materials, i.e. three, four, or more, in a single dosage form are additionally contemplated as within the scope of the presently claimed invention. In a preferred embodiment, at least one of these iron-providing materials contains a modified release mechanism, matrix, or coating. In a particularly preferred embodiment, one of the iron-providing materials provides an immediate, or non-modified, rate of release. In yet another preferred embodiment, the iron-providing materials naturally have different rates of release. Accordingly, each of the iron-providing material included within the composition has a different rate of release. Following administration to the animal, the iron-providing materials are released in the gastrointestinal tract over a period of up to 24 hours.

In a preferred, non-limiting aspect of the present inventive subject matter, the iron-providing materials are selected from the group consisting of carbonyl iron, soluble iron salts, slightly soluble iron salts, insoluble iron salts, chelated iron, and iron complexes.

In a preferred, non-limiting aspect of the present inventive subject matter, the soluble iron salts are selected from the group consisting of ferric hypophosphite, ferric albuminate, ferric chloride, ferric citrate, ferric oxide saccharated, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

In another preferred, non-limiting aspect of the present inventive subject matter, the slightly soluble iron salts are selected from the group consisting of ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

In yet another preferred, non-limiting aspect of the present inventive subject matter, the insoluble iron salts are selected from the group consisting of ferric sodium pyrophosphate, ferrous carbonate, ferric hydroxide, ferrous oxide, ferric oxyhydroxide, ferrous oxalate, other pharmaceutically acceptable iron salts and combinations thereof.

In still yet another preferred, non-limiting aspect of the present inventive subject matter, the iron complexes are selected from the group consisting of polysaccharide-iron complex, methylidine-iron complex, EDTA-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N′-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, MGD-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-PDTA ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

The use of different iron salts in the same dosage form, each having a different solubility profile, maximizes the rate and extent of absorption of iron. Since the different iron salts have different solubilities, the use of different iron sources allows multiple opportunities for iron uptake. This results in a reduction of the frequency of administration and the maintenance of effective concentrations of the biologically active substance in the blood. The maintenance of an effective concentration in the blood reduces the chance of side effects, such as nausea, and toxicity as occurs if too much of the biologically active substance is concentrated in the blood at any one time.

Another advantage to using at least two different iron-providing materials, each having a different solubility, is discovered when one considers that these materials are delivered to a changing gastric environment as the stomach cycles between the fasted and fed states. As the gastric environment changes between the fasted and fed states, the dissolution of a drug, vitamin, or mineral, and subsequent absorption in the small intestine, will vary, sometimes significantly. The key gastric environmental parameters in determining the extent of drug dissolution and subsequent absorption include pH, volume of gastric fluids, and GI motility patterns.

To enhance the rate and extent of dissolution and absorption of iron from the fasted and fed gastric environments, it is beneficial to use a combination of immediate and modified release iron salts and a combination of iron salts with different solubility profiles.

Iron absorption depends on the solubilization of iron in the acid milieu of the stomach and the interaction with other meal components in the lumen of the upper small intestine. The inclusion of immediate-release iron salts will provide for absorption of iron within the first hour of administration and beyond. The inclusion of mixed salts of iron will enhance the rate and extent of iron dissolution and absorption due to their varying solubilities. The inclusion of modified release iron salts will enhance the extent of iron dissolution and absorption as the gastric environment alternates between the fasted and fed states. Accordingly, it is advantageous to administer different iron sources having different solubility profiles.

In a key aspect of the present inventive subject matter, at least one of the iron-providing materials, i.e. carbonyl iron, soluble iron salts, slightly soluble iron salts, insoluble iron salts, chelated iron, and iron complexes, contains a modified release mechanism, matrix or coating. The modified release mechanism, matrix, or coating may be selected from the group consisting of polymeric and non-polymeric materials. Preferred polymeric pharmaceutically acceptable materials include but are not limited to ethylcellulose, methylcellulose, hydroxypropylmethylcellulose, cellulose acetate, hydroxypropylcellulose, hydroxyethylcellulose, carboxymethylcellulose, sodium carboxymethylcellulose, cellulose acetate phthalate, gelatin, polymethacrylates, polyvinyl alcohol, acrylic resins, other suitable polymers, and mixtures thereof.

In another preferred embodiment of the presently claimed invention, the modified release mechanism, matrix, or coating is composed of a synthetic or natural wax, fat, resin, or mixtures thereof. Preferred pharmaceutically acceptable waxes, fats, or resins include but are not limited to microcrystalline waxes, paraffin waxes, carnauba waxes, fatty acids and their salts, mono and diglyceride salts, esters of mono and diglycerides, agars, agaroses, algins, low methoxy pectins, gellans, K-carrageenan, t-carrageenan, furcellaran, β-carrageenan, curdlan, chitosan, konjac glucomannan and derivatives thereof including heat stable cold-melt knjac glucomannan, cellulose derivatives, starches, and mixtures of two or more of the foregoing, as well as hydrocolloid mixtures such as xanthan/locust bean gum, locust bean gumlagar, cassia/agar, cassia/xanthan, konjac/xanthan, carrageenan/locust bean gum, konjac/carrageenan, konjac/starch, other suitable waxes, fats, or resins, and mixtures thereof.

One non-limiting way to produce the modified release iron-providing material used in the present compositions is to disperse this material in a matrix by first liquifying the matrix material with heat and then dispersing the iron-providing material with a shearing or mixing operation. The iron-providing material remains dispersed in the matrix material as the matrix material solidifies or congeals. The iron-providing material may also be mixed with matrix forming excipients by micronizing the excipients and the iron-providing material prior to dry blending, followed by compression to form tablets or encapsulation into capsule shells.

In a preferred embodiment of the compositions of the presently claimed invention, the iron-providing materials are combined in an oral dosage form. A preferred, non-limiting example of an oral dosage form is a tablet, capsule, hard gel capsule, soft gel capsule, elixir, syrup, oral solution or suspension, chewable, soft gel, caramel, quick-dissolve, nutritional bar, sprinkle, or any other available oral dosage form known to a person of ordinary skill in the art of pharmaceutical administration technology. The oral dosage form may be administered to the patient once, twice, or thrice daily. Preferably the oral dosage form is administered once per day.

Compositions intended for oral use may be prepared according to any method known in the art for the manufacture of pharmaceutical compositions. In a preferred embodiment, the oral dosage form may contain additional materials selected from the group consisting of non-toxic carriers, binders, plasticizers, inert spherical substrate particles, matrix forming excipients, and sweetening agents.

In a preferred embodiment, the non-toxic carriers are selected from the group consisting of sugar, lactose, gelatin, starch, silicon dioxide, and mixtures thereof.

In another preferred embodiment, the binders are selected from the group consisting of povidone, pharmaceutical glaze, sugar, hydroxypropylmethyl cellulose, hydroxypropylcellulose, ethylcellulose, acrylic and methacrylic acid co-polymers, and mixtures thereof.

In still another preferred embodiment, the plasticizers are selected from the group consisting of diethyl phthalate, diethyl sebacate, triethyl citrate, crotonic acid, propylene glycol, castor oil, citric acid esters, dibutyl phthalate, dibutyl sebacate, polyethylene glycols, benzyl benzoate, glycerin, sorbitol, tributyl citrate, acetyltriethyl citrate, glyceryl triacetate, glyceryl tributarate, glyceryl diacetate, acetylated monoglycerides, other excipients, and mixtures thereof.

In yet another preferred embodiment, the inert spherical substrate particles are selected from the group consisting of sugar spheres and non-toxic plastic resin beads.

The oral dosage forms according to the present inventive subject matter may additionally contain one or more agents selected from the group consisting of a sweetening agent such as sucrose, lactose, or saccharin; flavoring agents such as peppermint, oil of wintergreen, or cherry; coloring agents; and preserving agents in order to provide pharmaceutically elegant and palatable preparations.

One embodiment of the oral dosage forms of the presently claimed invention is a tablet. Tablets containing the active ingredient in admixture with non-toxic pharmaceutically acceptable excipients may be manufactured by known methods.

In some cases, the oral dosage formulations may be in the form of hard gelatin capsules wherein the active ingredient is mixed with an inert solid diluent, for example calcium carbonate, calcium phosphate, or kaolin.

The present oral dosage formulations may also be in the form of soft gelatin capsules wherein the active ingredient is mixed with water or an oil medium, for example peanut oil, liquid paraffin, or olive oil.

Another embodiment of the oral dosage forms of the presently claimed invention is an oral solution or suspension. Drinking a composition suspended in a liquid is often easier and more pleasant for most patients than swallowing a pill. Because of the absence of carbonation, non-effervescent suspensions are easier to ingest than effervescent suspensions, especially if the patient is either experiencing nausea or is predisposed to nausea.

These solutions may be formed upon stirring, mixing or blending a solid dispersible tablet containing the iron-providing materials in a liquid. The solution may also be formed without stirring, mixing or blending the liquid after a solid dispersible tablet is placed in said liquid.

The use of a solution may provide improved patient compliance. Because of the presence of a flavoring and/or coloring agent, the present inventive subject matter has a palatable taste. Furthermore, because the present inventive subject matter is dispersed evenly throughout the liquid when it disintegrates, gastric discomfort associated with concentrated potassium chloride dosages are reduced or eliminated.

Exemplary non-limiting liquids in which the tablet may be placed in solution include water, milk, juices, or mixtures and combinations thereof. The liquid is preferably water.

Another embodiment of the oral dosage forms of the presently claimed invention is a chewable. Chewables containing the active ingredient in admixture with non-toxic pharmaceutically acceptable excipients may be manufactured by known methods.

The presently claimed invention additionally may contemplate the use of “other excipients” which may be prepared from a wide range of materials. Without being limited thereto, such materials include diluents, binders and adhesives, lubricants, plasticizers, disintegrants, colorants, bulking substances, flavorings, sweeteners, and miscellaneous materials such as buffers and adsorbents in order to prepare a particular medicated composition.

Exemplary non-limiting diluents which are of use as other excipients according to the present inventive subject matter may be selected from the group consisting of calcium phosphate, calcium sulfate, carboxymethylcellulose calcium, cellulose, cellulose acetate, dextrates, dextrin, dextrose, fructose, glyceryl palmitostearate, hydrogenated vegetable oil, kaolin, lactitol, lactose, magnesium carbonate, magnesium oxide, maltitol, maltodextrin, maltose, microcrystalline cellulose, polymethacrylates, powdered cellulose, pregelatinized starch, silicified microcrystalline cellulose, sodium chloride, sorbitol, starch, sucrose, sugar, talc, hydrogenated vegetable oil, and mixtures thereof.

Exemplary non-limiting binders which are of use as other excipients according to the present inventive subject matter may be selected from the group consisting of acacia, alginic acid, carboxymethylcellulose, hydroxyethylcellulose, hydroxypropylcellulose, dextrin, ethylcellulose, gelatin, liquid glucose, hydrogenated vegetable oil, hydroxypropylmethylcellulose, magnesium aluminum silicate, maltodextrin, methylcellulose, polyethylene oxide, polymethacrylates, povidone, sodium alginate, starch, zein, acrylic and methacrylic acid co-polymers, pharmaceutical glaze, gums such as guar gum, and milk derivatives such as whey and starches, as well as other conventional binders well known to persons skilled in the art.

Exemplary non-limiting lubricants which are of use as other excipients according to the present inventive subject matter may be selected from the group consisting of calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, sterilizable corn starch, talc, zinc stearate, and mixtures thereof.

Exemplary non-limiting plasticizers which are of use as other excipients according to the present inventive subject matter may be selected from the group consisting of lanolin, mineral oil, petrolatum, benzyl phenylformate, chlorobutanol, diethyl phthalate, glycerol, polyethylene glycol, sorbitol, triacetin, diethyl sebacate, triethyl citrate, cronotic acid, propylene glycol, butyl phthalate, dibtuyl sebacate, castor oil, and mixtures thereof. As is evident, the plasticizers may be hydrophobic as well as hydrophilic in nature.

Exemplary non-limiting disintegrants which are of use as other excipients according to the present inventive subject matter may be selected from the group consisting of alginic acid, carboxymethylcellulose, hydroxypropyl cellulose, microcrystalline cellulose, colloidal silicon dioxide, croscarmellose sodium, crospovidone, magnesium aluminum silicate, methylcellulose, polacrilin, povidone, sodium alginate, sodium starch glycolate, starch, and mixtures thereof.

Exemplary non-limiting colorants which are of use as other excipients according to the present inventive subject matter may be selected from the group consisting of curcumin, lactoflavin (riboflavin), tartrazine, quinoline yellow, sunset yellow FCF, cochineal carminic acid, carmoisine, ponceau 4R, patent blue V, indigo carmine, chlorophylls, lissamine green, caramel, black PN, carbo medicinalis vegetabilis, carotenoids, xanthophylls, betanin, anthocyanins, calcium carbonate, titanium dioxide, iron oxides and hydroxides, indigotine, alphazurine FG, indanthrene blue, fast green FCF, alizarin cyanine, quinizarine green SS, pyranine concentrated, orange II, dibromofluorescein, diiodofluorescein, erythrosine, ponceau SX, lithol rubin B, toney red, tetrabromofluorescein, eosine tetrachlorotetrabromofluorescein, phloxine B, helindone pink CN, brilliant lake red R, acid fuchsine, lake bordeaux B, flaming red, alba red, allura red AC, alizurol purple SS, tartrazine, sunset yellow FCF, fluorescein, naphthol yellow S, uranine, quinoline yellow, alumina, aluminum powder, annatto extract, beta-carotene, bismuth oxychloride, bronze powder, calcium carbonate, canthaxanthin, chromium-cobalt-aluminum oxide, chromium hydroxide green, cochineal extract, copper powder, dihydroxy acetone, ferric ammonium citrate, ferric ammonium ferrocyanide, ferric ferrocyanide, guanine, iron oxides synthetic, logwood extract, mica, potassium sodium copper chlorophyllin, pyrogallol, pyrophyllite, talc, zinc oxide, and mixtures thereof.

Exemplary non-limiting bulking substances which are of use as other excipients according to the present inventive subject matter may be selected from the group consisting of sugar, lactose, gelatin, starch, silicon dioxide, and mixtures thereof.

Exemplary non-limiting flavorings which are of use as other excipients according to the present inventive subject matter may be selected from the group consisting of ethyl maltol, fructose, maltol, tartaric acid, ethyl vanillin, fumaric acid, malic acid, menthol, vanillin, peppermint, oil of wintergreen or cherry, and mixtures thereof.

Exemplary non-limiting sweeteners which are of use as other excipients according to the present inventive subject matter may be selected from the group consisting of acesulfame potassium, aspartame, dextrose, fructose, liquid glucose, glycerol, lactitol, lactose, maltitol, maltose, saccharin, saccharin sodium, sodium cyclamate, sorbitol, sucrose, confectioner's sugar, xylitol, and mixtures thereof.

Preferably, the iron-providing materials are released from the present inventive compositions in the gastrointestinal tract of a patient for a period of up to 24 hours. In a preferred embodiment, the iron-providing materials are released in the gastrointestinal tract over a period of about 2 hours to about 18 hours.

It is of significant advantage to both the clinician and the patient that the supplement be formulated so that it may be administered in a minimum number of daily doses from which the drug is uniformly released over a desired, extended period of time. Often, the effectiveness of pharmaceuticals has a maximum life of a few hours in the body. As a result, the amount of active substance in the body fluctuates as the patient administers the composition every few hours, rather than remaining constant. Modified release dosage forms have modified release effects because of certain pharmaceutical excipients in the dosage form. A modified release dosage form can have a modified effect ranging from 1 hour to 1 week. By administering the active substance in a modified release form, patients do not need to purchase and administer as many doses, which will result in higher patient compliance. Further, by administering exactly the desired amount of active substance, nothing is wasted, and the composition is cost effective.

Since individual subjects may present a wide variation in severity of symptoms and each drug has its unique therapeutic characteristics, it is up to the practitioner to determine a subject's response to treatment and vary the dosages accordingly.

In a preferred aspect of the presently claimed invention, the amount of iron present in each dosage administered to a patient falls within the range of from about 5 mg to about 100 mg. In a particularly preferred aspect of the presently claimed invention, the amount of iron present in each dosage is from about 14 mg to about 90 mg.

The present inventive subject matter also includes methods for the enhancement of iron uptake or the treatment of iron deficiency in an animal in need thereof, which comprises: administering to said subject a therapeutically effective amount of at least two iron-providing materials combined in a single dosage form. The animal may be a human. Furthermore, the human may be an adult. Alternatively, the human may be a child. At least one of the iron-providing materials used in these methods contains a modified release mechanism, matrix, or coating. Each of the iron-providing materials used according to these methods has a different rate of release. Following administration to the animal, the iron-providing materials are released in the gastrointestinal tract over a period up to 24 hours.

The foregoing is considered as illustrative only of the principles of the invention. Further, since numerous modifications and changes will readily occur to those skilled in the art, it is not desired to limit the invention to the exact construction and operation shown and described. Accordingly, all suitable modifications and equivalents may be resorted to, falling within the scope of the invention.

### EXAMPLES

The following compositions were used to prepare the tablets for the enhancement of iron uptake or the treatment of iron deficiency according to the presently claimed invention:

### Example I

TABLE I

| | |
|---|---|
| Ingredient | Amount/Tablet |

| | | |
|---|---|---|
| Iron (Ferrous Fumarate, 90%) | 20.0 | mg |
| Vitamin E (di-alpha-Tocopheryl Acetate) | 30 | IU |
| Vitamin C (Ester-C Calcium Ascorbate) | 60.0 | mg |
| Thiamine (Thiamine Mononitrate, USP) | 3.0 | mg |
| Riboflavin (Riboflavin, USP) | 3.4 | mg |
| Pyridoxine (Pyridoxine HCl, USP) | 50.0 | mg |
| Cyanocobalamin | 12.0 | mcg |
| Niacin (Niacinamide, USP) | 20.0 | mg |
| Folic Acid (Folic Acid, USP) | 1.0 | mg |
| Calcium(Calcium Carbonate, Destab 38% Ca) | 200.0 | mg |
| Iron (Carbonyl Iron, 70% MicroMask) | 10.0 | mg |
| Copper (Cupric Oxide) | 2.0 | mg |
| Zinc (Zinc Oxide, Anhydrous) | 15.0 | mg |
| Magnesium (Magnesium Oxide, USP) | 100.0 | mg |

The formula described in this example was produced by the following method:

The iron salt is added to a melted wax or fatty acid in the molten form. The material is allowed to solidify. The solidified product is then milled. The milled product is added with the remaining ingredients and mixed together. The resulting composition is compressed into tablets.

### Example II

The method of producing Example I may be used to produce tablets for the enhancement of iron uptake or the treatment of iron deficiency according to the following formula:

TABLE II

| | |
|---|---|
| Ingredient | Amount/Tablet |

| | | |
|---|---|---|
| Vitamin A Acetate, USP (325 N IU/g) | 750.0 | IU |
| Beta-Carotene (167,000 Vitamin A IU/g) | 250.0 | IU |
| Cholecalciferol | 400.0 | IU |
| di-alpha-Tocopheryl Acetate | 11.0 | IU |
| Ascorbic Acid | 120.0 | mg |
| Folic Acid (Folic Acid, USP) | 1.0 | mg |
| Thiamine Mononitrate, USP | 2.0 | mg |
| Ribloflavin, USP | 3.0 | mg |
| Niacinamide, USP | 20.0 | mg |
| Pyridoxine Hcl, USP | 10.0 | mg |
| Cyanocobalamin 1.0% | 12.0 | mcg |
| Carbonyl Iron (Micromask 70%) | 35.0 | mg |
| Ferrous Sulfate, USP | 25.0 | mg |

### Example III

The method of producing Example I may be used to produce tablets for the enhancement of iron uptake or the treatment of iron deficiency according to the following formula:

TABLE III

| | |
|---|---|
| Ingrediant | Amount/Tablet |

| | | |
|---|---|---|
| Ferrous Furnarate | 36 | mg |
| Beta Carotene 10% | 1000 | IU |
| Cholecalciferol | 400.0 | IU |
| di-alpha-Tocopheryl Acetate | 11.0 | IU |
| Folic Acid, USP | 1.0 | mg |
| Thiamine Mononitrate | 2.0 | mg |
| Ribloflavin | 3.0 | mg |
| Niacinamide | 20.0 | mg |
| Pyridoxine HCl | 10.0 | mg |
| Sodium Ascorbate/Ascorbic Acid | 120.0 | mg |
| Cyanocobalamin | 12.0 | mcg |
| MicroMask Carbonyl Iron 70% | 36.0 | mg |

Chewable tablets incorporating the above formulations were prepared using conventional methods and materials known in the pharmaceutical art in either modified or immediate release forms. The resulting chewable tablets were recovered and stored for future use.

### Example IV

A patient is suffering from iron deficiency. A tablet in accordance with either Example I or II is administered, providing an enhanced iron uptake. It would be expected that the patient would improve his/her condition or recover.

### Example V

A patient is suffering from iron deficiency. A chewable tablet in accordance with Example III is administered, providing an enhanced iron uptake. It would be expected that the patient would improve his/her condition or recover.

### Example VI

A panel of six patients are put on a dosage regimen, but each patient is given a different dosage form (i.e. capsule or solid tablet of Example I). It would be expected that the patient with the highest compliance with the dosage regimen would be the patient taking the solid tablet of Example I.

The invention being thus described, it will be apparent that the same may be varied in many ways. Such variations are not to be regarded as a departure from the spirit and scope of the invention, and all such modifications are intended to be within the scope of the following claims.

We claim:

1. A composition for the enhancement of iron uptake or the treatment of iron deficiency by enhancing the rate and extent of iron dissolution in a subject in need thereof, which comprises: a combination of at least two iron-providing materials in a single dosage form;
wherein at least one of the iron-providing materials has a modified release mechanism, matrix, or coating;
wherein each of the iron-providing material has a different rate of release; and
wherein the iron-providing materials are released in the gastrointestinal tract over a period of up to 24 hours.

2. The composition of claim 1, wherein the iron-providing materials are selected from the group consisting of carbonyl iron, soluble iron salts, slightly soluble iron salts, insoluble iron salts, chelated iron, and iron complexes.

3. The composition of claim 2, wherein the carbonyl iron contains a modified release mechanism, matrix, or coating.

4. The composition of claim 1, wherein the modified release mechanism, matrix or coating is selected from the group consisting of ethylcellulose, methylcellulose, hydroxypropylmethylcellulose, cellulose acetate, hydroxypropylcellulose, hydroxyethylcellulose, carboxymethylcellulose, sodium carboxymethylcellulose, cellulose acetate phthalate, gelatin, polymethacrylates, polyvinyl alcohol, acrylic resins, other suitable polymers, and mixtures thereof.

5. The composition of claim 2, wherein the soluble iron salts are selected from the group consisting of ferric hypophosphite, ferric albuminate, ferric chloride, ferric citrate, ferric oxide saccharated, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

6. The composition of claim 5, wherein the soluble iron salts contain a modified release mechanism, matrix, or coating.

7. The composition of claim 2, wherein the slightly soluble iron salts are selected from the group consisting of ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

8. The composition of claim 7, wherein the slightly soluble iron salts contain a modified release mechanism, matrix, or coating.

9. The composition of claim 2, wherein the insoluble iron salts are selected from the group consisting of ferric sodium pyrophosphate, ferrous carbonate, ferric hydroxide, ferrous oxide, ferric oxyhydroxide, ferrous oxalate, other pharmaceutically acceptable iron salts and combinations thereof.

10. The composition of claim 9, wherein the insoluble iron salts contain a modified release mechanism, matrix, or coating.

11. The composition of claim 2, wherein the iron complexes are selected from the group consisting of polysaccharide-iron complex, methylidine-iron complex, EDTA-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N′-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, MGD-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-PDTA ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

12. The composition of claim 11, wherein the iron complexes contain a modified release mechanism, matrix, or coating.

13. The composition of claim 1, wherein the modified release mechanism, matrix, or coating is selected from the group consisting of microcrystalline waxes, paraffin waxes, carnauba waxes, fatty acids and their salts, mono and diglyceride salts, esters of mono and diglycerides, agars, agaroses, algins, low methoxy pectins, gellans, K-carrageenan, t-carrageenan, furcellaran, β-carrageenan, curdlan, chitosan, konjac glucomannan and derivatives thereof including heat stable cold-melt knjac glucomannan, cellulose derivatives, starches, and mixtures of two or more of the foregoing, as well as hydrocolloid mixtures such as xanthan/locust bean gum, locust bean gumlagar, cassia/agar, cassia/xanthan, konjac/xanthan, carrageenan/locust bean gum, konjac/carrageenan, konjac/starch, other suitable waxes, fats, or resins, and mixtures thereof.

14. The composition of claim 1, wherein the iron-providing materials are combined in an oral dosage form.

15. The composition of claim 14, wherein the oral dosage form is selected from the group consisting of a tablet, capsule, hard-gel capsule, soft-gel capsule, elixir, syrup, oral solution or suspension, chewable, soft gel, caramel, quick-dissolve, nutritional bar, and sprinkle.

16. The composition of claim 1, wherein the iron-providing materials are released in the gastrointestinal tract over a period of about 2 hours to about 18 hours.

17. The composition of claim 14, wherein the oral dosage form is administered once, twice, or thrice daily.

18. The composition of claim 14, wherein the oral dosage form contains additional materials selected from the group consisting of non-toxic carriers, binders, plasticizers, inert spherical substrate particles, matrix forming excepients, and sweetening agents.

19. The composition of claim 18, wherein the non-toxic carriers are selected from the group consisting of sugar, lactose, gelatin, starch, silicon dioxide, and mixtures thereof.

20. The composition of claim 18, wherein the binders are selected from the group consisting of povidone, pharmaceutical glaze, sugar, hydroxypropylmethylcellulose, hydroxypropylcellulose, ethylcellulose, acrylic and methacrylic acid co-polymers, and mixtures thereof.

21. The composition of claim 18, wherein the plasticizers are selected from the group consisting of diethyl phthalate, diethyl sebacate, triethyl citrate, crotonic acid, propylene glycol, castor oil, citric acid esters, dibutyl phthalate, dibutyl sebacate, polyethylene glycols, benzyl benzoate, glycerin, sorbitol, tributyl citrate, acetyltriethyl citrate, glyceryl triacetate, glyceryl tributarate, glyceryl diacetate, acetylated monglycerides, other excipients, and mixtures thereof.

22. The composition of claim 18, wherein the inert spherical substrate particles are selected from the group consisting of sugar spheres, and non-toxic plastic resin beads.

23. The composition of claim 1, wherein the composition comprises more than two iron-providing materials.

24. A composition for the enhancement of iron uptake or the treatment of iron deficiency in a mammal in need thereof, which comprises: a combination of at least two iron-providing materials in a single dosage form;
wherein at least one of the iron-providing materials has a modified release mechanism, matrix, or coating;
wherein each of the iron-providing material has a different rate of release; and
wherein the iron-providing materials are released in the gastrointestinal tract over a period of up to 24 hours.

25. A method for the enhancement of iron uptake or the treatment of iron deficiency in a subject in need thereof, which comprises: administering to said subject a therapeutically effective amount of at least two iron-providing materials combined in a single dosage form;
wherein at least one of the iron-providing materials has a modified release mechanism, matrix, or coating;
wherein each of the iron-providing material has a different rate of release; and
wherein the iron-providing materials are released in the gastrointestinal tract over a period of up to 24 hours.

26. The method of claim 25, wherein the iron-providing materials are selected from the group consisting of carbonyl iron, soluble iron salts, slightly soluble iron salts, insoluble iron salts, chelated iron, and iron complexes.

27. The method of claim 26, wherein the carbonyl iron contains a modified release mechanism, matrix, or coating.

28. The method of claim 25, wherein the modified release mechanism, matrix, or coating is selected from the group consisting of ethylcellulose, methylcellulose, hydroxypropylmethylcellulose, cellulose acetate, hydroxypropylcellulose, hydroxyethylcellulose, carboxymethylcellulose, sodium carboxymethylcellulose, cellulose acetate phthalate, gelatin, polymethacrylates, polyvinyl alcohol, acrylic resins, any other suitable polymers, and mixtures thereof.

29. The method of claim 26, wherein the soluble iron salts are selected from the group consisting of ferric hypophosphite, ferric albuminate, ferric chloride, ferric citrate, ferric oxide saccharated, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

30. The method of claim 29, wherein the soluble iron salts contain a modified release mechanism, matrix, or coating.

31. The method of claim 26, wherein the slightly soluble iron salts are selected from the group consisting of ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

32. The method of claim 31, wherein the slightly soluble iron salts contain a modified release mechanism, matrix, or coating.

33. The method of claim 26, wherein the insoluble iron salts are selected from the group consisting of ferric sodium pyrophosphate, ferrous carbonate, ferric hydroxide, ferrous oxide, ferric oxyhydroxide, ferrous oxalate, other pharmaceutically acceptable iron salts and combinations thereof.

34. The method of claim 33, wherein the insoluble iron salts contain a modified release mechanism, matrix, or coating.

35. The method of claim 26, wherein the iron complexes are selected from the group consisting of polysaccharide-iron complex, methylidine-iron complex, EDTA-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N′-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, MGD-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-PDTA ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

36. The method of claim 35, wherein the iron complexes contain a modified release mechanism, matrix, or coating.

37. The method of claim 25, wherein the modified release mechanism, matrix, or coating is selected from the group consisting of microcrystalline waxes, paraffin waxes, carnauba waxes, fatty acids and their salts, mono and diglyceride salts, esters of mono and diglycerides, agars, agaroses, algins, low methoxy pectins, gellans, K-carrageenan, t-carrageenan, furcellaran, β-carrageenan, curdlan, chitosan, konjac glucomannan and derivatives thereof including heat stable cold-melt knjac glucomannan, cellulose derivatives, starches, and mixtures of two or more of the foregoing, as well as hydrocolloid mixtures such as xanthan/locust bean gum, locust bean gumlagar, cassia/agar, cassia/xanthan, konjac/xanthan, carrageenan/locust bean gum, konjac/carrageenan, konjac/starch, other suitable waxes, fats, or resins, and mixtures thereof.

38. The method of claim 25, wherein the iron-providing materials are combined in an oral dosage form.

39. The method of claim 38, wherein the oral dosage form is selected from the group consisting of a tablet, capsule, hard gel capsule, soft gel capsule, elixir, syrup, oral solution or suspension, chewable, soft gel, caramel, quick-dissolve, nutritional bar, and sprinkle.

40. The method of claim 25, wherein the iron-providing materials are released in the gastrointestinal tract over a period of about 2 hours to about 18 hours.

41. The method of claim 38, wherein the oral dosage form is administered once, twice, or thrice daily.

42. The method of claim 38, wherein the oral dosage form contains additional materials selected from the group consisting of non-toxic carriers, binders, plasticizers, inert spherical substrate particles, matrix forming excipients, and sweetening agents.

43. The method of claim 42, wherein the non-toxic carriers are selected from the group consisting of sugar, lactose, gelatin, starch, silicon dioxide, and mixtures thereof.

44. The method of claim 42, wherein the binders are selected from the group consisting of povidone, pharmaceutical glaze, sugar, hydroxypropylmethylcellulose, hydroxypropylcellulose, ethylcellulose, acrylic and methacrylic acid co-polymers, and mixtures thereof.

45. The method of claim 42, wherein the plasticizers are selected from the group consisting of diethyl phthalate, diethyl sebacate, triethyl citrate, crotonic acid, propylene glycol, castor oil, citric acid esters, dibutyl phthalate, dibutyl sebacate, polyethylene glycols, benzyl benzoate, glycerin, sorbitol, tributyl citrate, acetyltriethyl citrate, glyceryl triacetate, glyceryl tributarate, glyceryl diacetate, acetylated monglycerides, other excipients, and mixtures thereof.

46. The method of claim 42, wherein the inert spherical substrate particles are selected from the group consisting of sugar spheres and non-toxic plastic resin beads.

47. The method of claim 25, wherein more than two iron-providing materials are combined in the single dosage form.

48. A method for the enhancement of iron uptake or the treatment of iron deficiency in a subject in need thereof, which comprises: administering to said subject a therapeutically effective amount of at least two iron-providing materials having different solubilities combined in a single dosage form;
wherein the iron-providing materials naturally have different rates of release; and
wherein the iron-providing materials are released in the gastrointestinal tract over a period of up to 24 hours.

49. The method of claim 48, wherein the iron-providing materials are selected from the group consisting of carbonyl iron, soluble iron salts, slightly soluble iron salts, insoluble iron salts, chelated iron, and iron complexes.

50. The method of claim 49, wherein the soluble iron salts are selected from the group consisting of ferric hypophosphite, ferric albuminate, ferric chloride, ferric citrate, ferric oxide saccharated, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

51. The method of claim 49, wherein the slightly soluble iron salts are selected from the group consisting of ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

52. The method of claim 49, wherein the insoluble iron salts are selected from the group consisting of ferric sodium pyrophosphate, ferrous carbonate, ferric hydroxide, ferrous oxide, ferric oxyhydroxide, ferrous oxalate, other pharmaceutically acceptable iron salts and combinations thereof.

53. The method of claim 49, wherein the iron complexes are selected from the group consisting of polysaccharide-iron complex, methylidine-iron complex, EDTA-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N′-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, MGD-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-PDTA ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

54. The method of claim 48, wherein the iron-providing materials are combined in an oral dosage form.

55. The method of claim 54, wherein the oral dosage form is selected from the group consisting of a tablet, capsule, hard gel capsule, soft gel capsule, elixir, syrup, oral solution or suspension, chewable, soft gel, caramel, quick-dissolve, nutritional bar, and sprinkle.

56. The method of claim 48, wherein the iron-providing materials are released in the gastrointestinal tract over a period of about 2 hours to about 18 hours.

57. The method of claim 54, wherein the oral dosage form is administered once, twice, or thrice daily.

58. The method of claim 54, wherein the oral dosage form contains additional materials selected from the group consisting of non-toxic carriers, binders, plasticizers, inert spherical substrate particles, matrix forming excipients, and sweetening agents.

59. The method of claim 58, wherein the non-toxic carriers are selected from the group consisting of sugar, lactose, gelatin, starch, silicon dioxide, and mixtures thereof.

60. The method of claim 58, wherein the binders are selected from the group consisting of povidone, pharmaceutical glaze, sugar, hydroxypropylmethylcellulose, hydroxypropylcellulose, ethylcellulose, acrylic and methacrylic acid co-polymers, and mixtures thereof.

61. The method of claim 58, wherein the plasticizers are selected from the group consisting of diethyl phthalate, diethyl sebacate, triethyl citrate, crotonic acid, propylene glycol, castor oil, citric acid esters, dibutyl phthalate, dibutyl sebacate, polyethylene glycols, benzyl benzoate, glycerin, sorbitol, tributyl citrate, acetyltriethyl citrate, glyceryl triacetate, glyceryl tributarate, glyceryl diacetate, acetylated monglycerides, other excipients, and mixtures thereof.

62. The method of claim 58, wherein the inert spherical substrate particles are selected from the group consisting of sugar spheres and non-toxic plastic resin beads.

63. The method of claim 48, wherein more than two iron-providing materials are combined in the single dosage form.
